(19) United States
(12) Patent Application Publication
(10) Pub. No.: US 2013/0028849 A1
(43) Pub. Date: Jan. 31, 2013

(54) Agent For Stimulating The Expression of Loxl

(71) Applicants: Valérie Cenizo, Lyon (FR); Nabil Abdul-Malak, Caluire (FR); Valérie Andre, Ampuis (FR)

(72) Inventors: Valérie Cenizo, Lyon (FR); Nabil Abdul-Malak, Caluire (FR); Valérie Andre, Ampuis (FR)

(73) Assignee: BASF Beauty Care Solutions France S.A.S., Lyon (FR)

(21) Appl. No.: 13/519,235

(22) PCT Filed: Dec. 23, 2010
(86) PCT No.: PCT/EP10/70662

§ 371 Date: Jun. 26, 2012

Related U.S. Application Data
(60) Provisional application No. 61/292,225, filed on Jan. 5, 2010.

(30) Foreign Application Priority Data
Dec. 31, 2009 (FR) 0959682

(51) Int. Cl. A61K 36/74; A61P 17/02; A61P 17/00; A61Q 19/08; A61P 39/06; A61P 17/14; A61P 9/10; A61P 3/10; A61P 25/00; A61P 35/00; A61P 17/06; A61K 8/97; A61P 9/00

(52) U.S. Cl. 424/58; 424/725; 424/774; 424/779; 424/64; 424/59

(57) ABSTRACT

The present invention concerns a novel agent which is capable of stimulating the expression of LOXL and of increasing quality and functionality of collagen fibres and has anti-oxidizing, anti-elastase, anti-MMP properties as well as its use in producing cosmetic or pharmaceutical compositions, especially dermatological compositions, or food compositions.

The present invention relates to a novel cosmetic and/or dermatological agent and to its use for the production of a composition, in particular cosmetic, food or pharmaceutical, in particular dermatological, especially intended to prevent and/or combat aging, preferably skin aging, in particular induced by ultraviolet radiation (UV).

Skin aging, in particular photo-induced aging, is a complex phenomenon involving several mechanisms which have been studied in detail, such as the production of reactive compounds, ROS (reactive oxygen species), in particular free radicals which are responsible for the phenomenon of oxidation (lipid peroxidation and protein oxidation) and for the production of enzymes such as elastases and metalloproteinases which are at the origin of the destruction of tissues and fibres such as collagens and elastin. Thus, those mechanisms are in general responsible for the disorganization of elastic tissues.

In particular, native collagen fibres are specifically degraded by metalloproteinases (MMP), in particular MMP-1, the production of which is induced from 0.001 MED (minimum erythema dose), i.e. equivalent to just 3 minutes of exposure to the sun. This MMP-1 production changes with the duration and intensity of exposure in a dose-dependent manner. Moreover, collagen synthesis is reduced under UV stress.

Loss of elasticity is one of the major problems with exposure to the sun, but more generally of skin aging. Elastin fibres, association with tropoelastin elements and microfibrills, in the dermis provide the elasticity functions. During aging, in particular under the action of a UV stress, the formation of elastic fibres decreases and its degradation by MMPs and elastases increases.

MMPs and ROS are also pro-inflammatory factors which contribute to skin damages.

These mechanisms have been investigated in great detail in the fields of cosmetics and dermatology and many anti-oxidizing agents, anti-MMP or anti-elastase agents are already on the market. Combinations of anti-elastase and anti-MMP agents have thus been described in order to prevent and repair UV damages. The active ingredients are therefore frequently combined into cosmetic compositions because then their properties are complementary. The search for compounds with complementary properties is thus of great interest in these fields.

More recently, the Applicant discovered that LOXL, an isoform enzyme from the lysyl oxidase family (LOX), was a principal chain link missing in elastogenesis in the adult and it was possible to reactivate the synthesis of this isoform of lysyl oxidase in order to obtain a stimulant effect on elastogenesis. The Applicant demonstrated, on a reconstructed skin model producing elastic fibres, that activation of the synthesis of LOXL re-established functional elastogenesis. The Applicant also demonstrated a deficit in the expression of LOXL in cicatricial regions, as well as in the dermis of the human skin at different ages, and thus during aging. LOXL is the enzyme responsible for maturing of elastin by cross-linking and thus allows the formation of functional elastic fibres. The Applicant has already identified the active agents allowing LOXL expression stimulation (patent applications FR 2 855 968 and FR 2 855 969, US 2004-0253220 and US 2004-0258676, incorporated herein in their entirety by reference).

The search for alternative active agents to those described in the prior art continues to be of great interest in the field of cosmetics and dermatology. Further, an active agent having several of the properties described above, in particular properties which both protect the elastic fibres and stimulate their synthesis, is particularly necessary.

The Applicant has now unexpectedly and surprisingly discovered a novel agent which satisfies this need in that it stimulates the synthesis of LOXL and has several additional protective properties of great interest in the cosmetics field and in the pharmaceutical field in order to prevent and/or combat aging, in particular skin aging. Indeed, the agent of the present invention has the property of stimulating the expression of LOXL and has anti-oxidizing and anti-elastase properties. It also has anti-MMP properties, in particular anti-MMP-1, anti-MMP-2, and anti-MMP-12. It was also discovered that agent of the invention is able to improve quality and/or functionality of collagen fibres, in particular type I collagen.

This agent of the invention is a new cosmetic, pharmaceutical, notably dermatological and/or food active ingredient which by itself can provide a complete, effective and thus entirely satisfactory solution to the problem of aging, in particular skin aging and in particular photo-induced aging. It may also be used in combination with other prior art agents in order to boost their properties. This agent can thus provide a particularly effective solution to the problem of the prior art.

The agent of the invention is a plant extract and thus has the advantage of being easy to produce on an industrial scale, of being easy to formulate and of having high stability. It is also readily available and extracted from a renewable source, meaning that it can be manufactured in accordance with the principles of sustainable development.

The agent of the invention is an extract from a plant belonging to the genus Geophila, in particular an extract from Geophila cordifolia and/or Geophila repens.

The genus Geophila of the invention designates the plant genus Geophila belonging to the Rubiaceae Juss. family according to the Angiosperm Phylogeny Group, APGII, 2003, classification. In particular, this genus includes Geophila cordifolia and/or Geophila repens.

Geophila cordifolia is also known as Geophila trichogyne, and Mapouria trichogyre is cultivated in South America. Broadly, Geophila cordifolia includes 3 types of varieties: Geophila cordifolia in the strict sense, Geophila cordifolia var. cordifolia and Geophila cordifolia var. peruviana.

Geophila repens (L.) I.M. Johnst. is also known as Geophila herbacea and also cultivated in South America.

None of the known properties of the genus Geophila, in particular Geophila cordifolia and/or Geophila repens, could be used to predict its interesting properties in the field of cosmetics or pharmaceuticals as has been discovered in the context of the present invention.

Certain properties of a plant extract from the genus Geophila, in particular an extract of Geophila cordifolia and/or Geophila repens, of the invention will be demonstrated in more detail in the examples supplied below regarding its radical scavenging activity, anti-MMP activity, anti-elastase activity and its stimulating effects on elastic and/or collagen fibres and on stimulation the expression of LOXL.

Thus, the present invention concerns the use of a plant extract from the genus Geophila, preferably from Geophila cordifolia and/or Geophila repens, as an agent for stimulating the expression of LOXL and/or an anti-oxidizing agent and/or an anti-elastase agent and/or an anti-MMP, in particular an anti-MMP-1, anti-MMP-2, anti-MMP-12 agent and/or for increasing the quality and/or the functionality of collagen fibres, in particular type I collagen in extracellular matrix.

The present invention also concerns the use of an extract from a plant from the genus Geophila, in particular Geophila cordifolia and/or Geophila repens, in a cosmetic, pharmaceutical, dermatological and/or food supplement composition, in particular as an active agent to prevent and/or combat aging in a human being, in particular skin aging, and/or an alteration in the elastic and/or collagen fibres.

The present invention also concerns cosmetic, dermatological, food supplement or pharmaceutical compositions comprising a plant extract from the genus Geophila, preferably Geophila cordifolia and/or Geophila repens, especially in combination with a vehicle suitable for cosmetic, dermatological, food supplement or pharmaceutical use.

The present invention also concerns a method or process of skin care and/or cosmetic treatment, comprising topical application or oral administration of a composition of the invention or of a plant extract from the genus Geophila, preferably Geophila cordifolia and/or Geophila repens, as described in the present description (above and below), in order to prevent and/or reduce and/or retard an alteration in elastic and/or collagen fibres, loss of elasticity and/or flexibility and/or firmness in the tissues, in particular the skin, and/or to prevent and/or reduce unattractive and/or disagreeable and/or uncomfortable manifestations linked to aging, in particular in the skin, and/or to stimulate the expression of LOXL and/or as a radical scavenger, an anti-elastase and/or anti-MMP agent, to increase the quality and/or the functionality of collagen fibres, in particular type I collagen and/or to prevent and/or reduce the unattractive and/or disagreeable and/or uncomfortable effects of skin aging.

### DEFINITION

In accordance with the invention, the term “LOXL” or “hLOXL”, also known as LOXL-1, means an isoform of the lysyl oxidase (LO) family, a family comprising 5 members: LOX, LOXL, LOXL-2, LOXL-3, LOXL-4 and in particular described in the publication by Csiszar et al. Lysyl oxidases: “A novel multifunctional amine oxidase family”, Nucleic Acid Research and Molecular Biology, 2001, vol 70, p2-28.

According to the invention, the term “stimulates the expression of LOXL” means stimulation of the expression of the gene encoding LOXL or its promoter, and in particular stimulation of the synthesis of the messenger RNA encoding LOXL, but also stimulation of the synthesis of LOXL from said messenger RNA.

According to the invention, the term “anti-elastase agent” means a product which is capable of inhibiting the enzymatic activity of at least one elastase, in particular human leucocyte elastase (HLE) and/or human pancreatic elastase.

According to the invention, the term “radical scavenger” means a product which can trap free radicals.

According to the invention, the term “anti-MMP agent” means an antagonist product to the synthesis and/or activity of matrix metalloproteinases, preferably the activity of MMPs, in particular MMP-1, MMP-2 and MMP-12.

According to the invention, the term “increase quality and/or functionality of collagen fibres” means increase fibrillar collagen content in extracellular matrix, in particular type I collagen, and/or increase thickness of collagen fibres, in particular type I collagen and/or decrease anisotropy in collagen fibres and/or stimulate synthesis of collagen by fibroblasts in particular proteic synthesis of protein. Collagen as mentioned in the present invention is preferably fibrillar collagen, preferably selected in the group of type 1, 3, 4 and 7. According to preferred embodiment, collagen is type 1 collagen.

The term “topical application” as used here means applying the composition of the present invention onto the surface of the skin and/or mucosae, in particular by direct application or by vaporization.

The agent of the invention may be extracted from the whole plant or from one or more parts of the plant, and in particular selected from the root, stem, bark, flower, germ, seed and/or leaf, and mixtures thereof. The agent of the invention is preferably extracted from the aerial parts of the plant, in particular the stem, leaves and a mixture thereof.

The extract may thus be obtained by plant extraction methods which are known in the field, for example by maceration of at least a portion of the plant, preferably at between 0.2% and 10% (w/w), preferably between 0.5% and 5% (w/w), more preferably around 1% (w/w), in a solvent or a mixture of solvents, preferably a polar protic solvent, and advantageously in water, an alcohol, a glycol, a polyol, a water/alcohol, water/glycol or water/polyol mixture (such as water mixed with ethanol, glycerol, butylene glycol or other glycols, such as xylitol etc) from 100/0 to 0/100 (v/v). The extracts obtained are then preferably centrifuged and/or filtered and/or distilled in order to recover the active soluble fraction (raw extract). Supplemental steps for bleaching and/or deodorizing may be carried out on the extract at any stage of the extraction, using techniques which are known to the skilled person.

The plant extract is preferably dissolved in a solvent, in particular a polar solvent such as water, an alcohol, a polyol, a glycol or a mixture thereof.

The active substance may then be concentrated by evaporating off the solvent, for example by lyophilization or spraying.

In accordance with an advantageous implementation, the agent of the invention is an extract obtained by cold and/or ambient temperature maceration, preferably a mixture of stems and leaves, optionally after a step for drying the plant. The extract is then dissolved in an aqueous vehicle, preferably water. The extract is then used in accordance with the present invention, optionally after filtering.

In a preferred embodiment, maceration is carried out at a temperature which is between 0° C. and 25° C., preferably between 4° C. and 20° C. Maceration period is preferably chosen between 30 min and 24 hours, and preferably between 2 and 10 hours.

In accordance with the invention, the plant extract from Geophila, preferably from Geophila cordifolia and/or Geophila repens, is preferably used alone or in a cosmetic, dermatological, food supplement or pharmaceutical composition at a concentration between 1×10⁻⁴ and 10% by weight, advantageously between 1×10⁻⁴ and 5%, and more particularly between 1×10⁻³ and 3% by weight with respect to the total composition weight.

The plant extract from Geophila, preferably from Geophila cordifolia and/or Geophila repens, can be used to stimulate the expression of LOXL and/or as a radical scavenger and/or as an anti-MMP agent and/or as an anti-elastase agent and/or to increase quality and/or functionality of collagen fibres, in particular type I collagen.

The plant extract from Geophila, preferably from Geophila cordifolia and/or Geophila repens, may thus be used alone or in a cosmetic or pharmaceutical composition to prevent and/or combat aging in the human being, in particular skin aging, and/or changes to elastic fibres, in particular the loss of their functions, especially their elasticity and/or loss of quality and/or functionality of collagen fibres.

The extract of the invention can prevent and/or repair unattractive, uncomfortable and/or disagreeable manifestations of skin aging, and/or to an alteration in the elastic and/or collagen fibres.

The unattractive, uncomfortable and/or disagreeable manifestations of aging and/or linked to an alteration in the elastic and/or collagen fibres results from a loss of elasticity, resistance, firmness and/or flexibility in the tissues. In the skin in particular, they are wrinkles, fine lines, skin described as being “shrunken”, “slack”, “thin”, with a dull complexion, bags, a reduction in vascularization such as couperose, rosacea, a loss of fatty tissue in particular in the hypodermis, unattractive, uncomfortable and/or disagreeable manifestations due to incomplete or imperfect elastogenesis such as unattractive scars and/or stretch-marks, or solar elastosis.

The agent of the invention is particularly suitable for preventing and/or combating unattractive, uncomfortable and/or disagreeable signs of chronobiological aging, also termed intrinsic aging or chronoaging, photo-induced aging, i.e. due to exposure to the sun and/or UV, and/or induced by aggressive factors, especially environmental agents, such as pollutants (tobacco, smoke), factors responsible for modifying the physiology of the skin such as emotional factors, especially stress, variations in pH such as perspiration, variations in temperature such as climatic factors (wind, cold, heat) and/or chemical agents (heavy metals, detergents, compounds contained in cosmetic treatments such as fragrances, preservatives, AHA alcohols or dermatological treatments, such as vitamin A acid) and/or aggressive conditions, in particular mechanical challenges such as depilation, shaving, rubbing.

Due to its ability to increase quality and/or functionality of collagen fibres, in particular type I collagen, the agent of the invention is particularly suitable for preventing and/or combating stretch-marks, unattractive, uncomfortable and/or disagreeable signs of scars.

In addition, the agent of the invention is particularly suitable for the care and/or treatment of sensitive, sensitized and/or reactive skin, specially after sun exposure.

Furthermore, the extract of the invention can prevent and/or treat pathologies linked to skin aging and/or to a loss of functionality of elastic and/or collagen fibres, in particular, in the case of skin tissues, fibrosis, scleroderma, atherosclerosis, emphysema, pseudo-xanthoma elasticum (PXE), hypertrophic scar, keloid scar, dystrophic scar and/or associated with a genetic deficiency affecting the elastic and/or collagen fibres, such as Marfan syndrome, progeria, cutis laxa, genetic solar elastosis, Ehler-danlos syndrom and/or pathologies linked to vascular tissue deficiency.

The extract of the invention can also prevent and/or treat pathologies due to an anti-oxidizing stress, such as cardiovascular diseases, atherosclerosis, diabetes, neuropathies or cancer.

The extract of the invention can also prevent and/or treat pathologies due to elastases and/or metalloproteinases, especially elastolysis affecting pulmonary tissue, arteries, gums and skin, especially due to diseases linked to a deficiency in elastase and/or metalloproteinase inhibitors, in particular serpins, cystatins and TIMPs (tissue inhibitor of metalloproteinases), varicose veins and inflammations, gingivitis and periodontitis, psoriasis, dermatitis and eczema.

As mentioned above, the extract from the plant from the genus Geophila, in particular Geophila cordifolia and/or Geophila repens, can be used alone. It is preferably used in the form of cosmetic or pharmaceutical compositions, preferably dermatological compositions.

Thus, the present invention provides a cosmetic, pharmaceutical and in particular dermatological composition containing an extract from a plant from the genus Geophila, and in particular Geophila cordifolia and/or Geophila repens, in combination with an appropriate cosmetic or pharmaceutical, especially dermatological, vehicle. Such a composition can be used to stimulate the expression of LOXL and/or as a radical scavenger and/or as an anti-MMP agent, in particular anti-MMP-2 and/or anti-MMP-12 agent and/or as an anti-elastase agent and/or increase quality and/or functionality of collagen fibres in particular type I collagen.

The cosmetic composition of the invention is thus particularly suitable for preventing and/or repairing unattractive, uncomfortable and/or disagreeable manifestations of skin aging and/or to an alteration in elastic and/or collagen fibres.

The pharmaceutical composition of the invention is thus particularly intended for the care and/or treatment of pathologies associated with aging, in particular skin aging, and/or with an alteration in elastic and/or collagen fibres.

The compositions of the invention may contain any appropriate solvent and/or any appropriate vehicle and/or any appropriate excipient, optionally in combination with other compounds of interest.

For this reason, for these compositions, the excipient contains, for example, at least one compound selected from the group consisting of preservatives, emollients, emulsifying agents, surfactants, moisturizers, thickening agents, conditioning agents, mattifying agents, stabilizing agents, anti oxidants, texturizing agents, gloss agents, film-forming agents, solubilizers, pigments, dyes, fragrances and sunscreens. These excipients are preferably selected from the group consisting of amino acids and their derivatives, polyglycerols, esters, polymers and cellulose derivatives, lanolin derivatives, phospholipids, lactoferrins, lactoperoxidases, sucrose-based stabilizers, E vitamins and their derivatives, natural and synthetic waxes, vegetable oils, triglycerides, non-saponifiables, phytosterols, plant esters, silicones and their derivatives, protein hydrolysates, jojoba oil and its derivatives, lipo/hydrosoluble esters, betaines, aminoxides, plant extracts, saccharose esters, titanium dioxides, glycines, and parabens, and more preferably from the group consisting of butylene glycol, steareth-2, steareth-21, glycol-15 stearyl ether, cetearyl alcool, phenoxyethanol, methylparaben, ethylparaben, propylparaben, butylparaben, butylene glycol, natural tocopherols, glycerin, dihydroxycetyl sodium phosphate, isopropyl hydroxycetyl ether, glycol stearate, triisononanoin, octyl cocoate, polyacrylamide, isoparaffin, laureth-7, a carbomer, propylene glycol, glycerol, bisabolol, a dimethicone, sodium hydroxide, PEG 30-dipolyhydroxysterate, capric/caprylic triglycerides, cetearyl octanoate, dibutyl adipate, grape seed oil, jojoba oil, magnesium sulphate, EDTA, a cyclomethicone, xanthan gum, citric acid, sodium lauryl sulphate, mineral oils and waxes, isostearyl isostearate, propylene glycol dipelargonate, propylene glycol isostearate, PEG 8, beeswax, hydrogenated palm kernel oil glycerides, hydrogenated palm oil glycerides, lanolin oil, sesame oil, cetyl lactate, lanolin alcohol, castor oil, titanium dioxide, lactose, saccharose, low density polyethylene, and isotonic saline solution.

Advantageously, the compositions cited above are formulated into a form selected from the group consisting of a solution, aqueous or oily, a cream or an aqueous or oily gel, in particular in a pot or a tube, especially a shower gel, a shampoo; a milk; an emulsion, a microemulsion or a nanoemulsion, especially oil-in-water or water-in-oil or multiple or siliconized; a lotion, in particular in a glass or plastic bottle or dispensing bottle or aerosol; an ampoule; a liquid soap; a dermatological cake; a ointment; a foam; an anhydrous product, preferably liquid, paste or solid, for example in the form of a stick; and powders.

Preferably, the cosmetic composition of the invention is in the form of an anti-wrinkle or anti-aging cream, in particular intended to be applied on skin termed “mature”, a composition for sensitive and/or irritated skin, a hair composition, in particular for anti-hair loss or for hair regrowth, a product for making up the skin of the face, body or lips such as a foundation, a lipstick, an oral hygiene product such as a toothpaste, or a mouthwash lotion.

In an advantageous embodiment, the cosmetic composition of the invention is a composition protecting skin against UV damages, notably a sunscreen composition and/or an after-sun care composition.

Preferably, the plant extract from the genus Geophila, preferably Geophila cordifolia and/or Geophila repens, of the invention, preferably in the form of a cosmetic composition of the invention, is applied to at least one zone of the body where it is desired to prevent and/or combat aging, in particular onto at least one zone of the body where it is unattractive, uncomfortable and/or disagreeable, this or these zones preferably being a surface of the body selected from the skin of the face, including the forehead, the contour of the lips and/or the contour of the eyes (peri-orbit), the oval of the face and the skin of the body including the neck, the hands, the arms, the thighs, the stomach, the hips and/or the bust.

In particular, the cosmetic compositions of the invention are intended for the care and/or cosmetic treatment of mature skin and/or skin with a sun-sensitive phototype.

Preferably, the extract from the plant from the genus Geophila, preferably Geophila cordifolia and/or Geophila repens, of the invention, preferably in the form of a pharmaceutical composition of the invention, is applied to at least one zone of the body having a pathology linked to skin aging and/or to an alteration in the elastic fibres and/or to an alteration in the collagen fibres, this or these zones preferably being a surface of the body selected from the skin of the face, including the forehead, the contour of the lips and/or the contour of the eyes (peri-orbit), the oval of the face and the skin of the body including the neck, the hands, the arms, the thighs, the stomach, the hips and/or the bust.

The cosmetic or pharmaceutical compositions of the present invention may be administered topically or orally.

The term “appropriate cosmetic or dermatological vehicle” used here means that the composition or the components thereof are suitable for use in contact with the human skin without undue toxicity, incompatibility, instability, allergic response, or their equivalents.

The term “appropriate pharmaceutical vehicle” used here means that the composition or the components thereof are suitable for use in contact with the human body without undue toxicity, incompatibility, instability, allergic response, or their equivalents.

Many cosmetically active ingredients are known to the skilled person for improving the health and/or physical appearance of the skin. The skilled person will know how to formulate the cosmetic or dermatological compositions in order to obtain the best effects. Further, the compounds described in the present invention may have a synergistic effect when they are combined with each other. These combinations are also covered by the present invention. The CTFA Cosmetic Ingredient Handbook, Second Edition (1992) describes various cosmetic and pharmaceutical ingredients which are routinely used in the cosmetics and pharmaceuticals industry which in particular are suitable for topical use. Non-limiting examples of these classes of ingredients include the following compounds: abrasives, absorbants, compounds with an aesthetic aim such as fragrances, pigments, dyes, essential oils, astringents, etc (for example: clove oil, menthol oil, camphor oil, eucalyptus oil, eugenol, menthyl lactate, witch hazel distillate), anti-acne agents, anti-flocculants, anti-foaming agents, antimicrobial agents (for example: iodopropyl butylcarbamate), anti-oxidants, binders, biological additives, buffers, swelling agents, chelating agents, additives, biocidal agents, denaturing agents, thickening agents, and vitamins, and their derivatives or equivalents, film-forming materials, polymers, opacifying agents, pH adjusters, reducing agents, de-pigmenting or brightening agents (for example: hydroquinone, kojic acid, ascorbic acid, magnesium ascorbyl phosphate, ascorbyl glucosamine), conditioning agents (for example: humectants).

Particularly advantageously, the extract from a plant from the genus Geophila, preferably Geophila cordifolia and/or Geophila repens, of the invention may optionally be used in a cosmetic or pharmaceutical composition, preferably dermatological, preferably those described above, as the sole active agent, in particular as the sole active agent stimulating the expression of LOXL, radical scavenger, anti-MMP and/or anti-elastase agent and/or to increase quality and/or functionality of collagen fibres or in combination with one of more other active agents selected from:

1) another agent of the same cosmetic and/or dermatological type, in particular:
Another MMP inhibitor, in particular from MMP-1, MMP-2, MMP-3, MMP-7, MMP-9, MMP-11, MMP-12, MMP-13, MMP-14, MMP-15, MMP-16 and MMP-17, especially a tissue inhibitor of metalloproteinases (TIMP), such as the peptides known in the art as TIMP-1, TIMP-2, TIMP-3 and TIMP-4, a MMP inhibitor such as ursolic acid, carotenoids, vitamin C, isoflavones such as genistein, lycopene, retinol, retinoic acid and their derivatives and/or a plant extract containing it, and/or a malt extract marketed by the Applicant under the trade name Collalift™;
another radical scavenging agent such as sodium sulfites, sodium disulfites vitamin E, bioflavonoids, Q10 coenzyme or ubiquinone, nordihydroguaiaretic acid and derivatives thereof and/or a plant extract containing it, and/or certain enzymes such as catalase, superoxide dismutase, lactoperoxidase, glutathion peroxidase and quinone reductases;
another elastase inhibiting agent such as micromerol;
another LOXL expression stimulating agent, in particular aneth extract described in patent application FR 2 855 968;
   and/or
   2) an agent with complementary properties:
an exfoliating and/or keratolytic agent, in particular alpha-hydroxy acids (AHA), in particular salicylic acid, optionally in combination with acacia proteins, malic acid, optionally in association with almond proteins, glycolic acid, lactic acid, and/or derivatives thereof, and/or mixtures thereof;
an agent stimulating fibronectin synthesis, in particular a corn extract, such an extract being marketed by the Applicant under the trade name Deliner™;
a fibroblast growth factor (FGF2) agent for protection of the extracellular matrix against its degradation and/or denaturing, especially an extract of Hibiscus Abelmoscus as described in the Applicant's patent application filed with number FR 0 654 316 and/or a fibroblast growth stimulation agent, for example a fermented soya extract containing peptides, known under the trade name Phytokinen™ marketed by the Applicant and also described in patent application EP 1 119 344 B1 (Laboratoires Expanscience), and preferably a combination of these two extracts as described in patent application WO;
an agent stimulating the synthesis of laminin, in particular a malt extract modified by biotechnology, such an extract being marketed by the Applicant under the trade name Basaline™;
an agent stimulating the expression and/or activity of hyaluronane synthase 2 (HAS2), such as plant extracts as described in patent application FR 2 893 252A1, in particular an aqueous extract of Galanga (Alpinia galanga);
a slimming agent, especially selected from the phosphodiestease enzyme inhibitor group, an adenylate cyclase activation agent, AMPc, preferably caffeine, forskolin, theophyllin, theobromine, in particular a mixture of caffeine, carnitine, Centella asiatica and esculoside sold under the trade name Sveltine™, or a solution of sulphated oligosaccharides sold by the Applicant under the trade name Slim-Excess™ and described in patent application WO 2009/000935;
a re-plumping agent, especially hyaluronic acid filling spheres sold by the Applicant under the trade name Hyaluronic Filling Spheres™;
a calming agent, especially an extract from Pueraria lobata roots sold by the Applicant under the trade name Inhipase™ and described in the patent application published with number FR2 847 267;
an anti-inflammatory agent, in particular cytokine and chemokine production inhibitors, cyclooxygenase production inhibitors, NO and NO-synthase inhibitors, especially extracts from Vitis vinifera, Gingko biloba extracts, trilactone terpenes such as gingkolides, in particular gingkolide B and bilobalide for their plate activation factor (PAF) property;
an anti-pollution agent, i.e. a compound capable of trapping ozone, mono- or poly-cyclic aromatic compounds such as benzopyrene and/or heavy metals such as cobalt, mercury, cadmium and/or nickel; such as vitamin C and derivatives thereof, phenols and polyphenols, in particular tannins, ellagic acid and tannic acid, epigallocatechin and natural extracts containing it; tea extracts, in particular from green tea, anthocyans, phenol acids, stilbenes, in particular resveratrol and derivatives thereof;
a glycation inhibiting agent such as those described in the Applicant's patent application WO 2009/007411, and/or a deglycation agent, such as those described in the Applicant's patent application WO 2009/007412;
an agent stimulating the synthesis of intracellular ATP, especially an extract from the algae Laminaria digitata;
an agent with general anti-aging activity, especially against pigmented marks, in particular niacinamide or vitamin B3;
UVA and/or UVB filters in the form of organic or inorganic compounds, such as oxides of zinc, iron, zirconium, cerium and/or titanium, ethylhexyl salicylate, ethylhexyl methoxycinnamate, butylmethoxydibenzoyl methane, possibly coated, emulsified or encapsulated, in particular in the form of a liposome.
an anti-stretch marks agent

The agent of the invention may also be administered in the form of and/or in association with a marine DNA microsphere encapsulating vitamin C and E derivatives which are liberated under UV radiation, especially those marketed under the trade name Smartvector UV™ and described in the Applicant's French patent application FR 2 848 854.

The present invention also pertains to a cosmetic care method in which the extract from a plant from the genus Geophila, in particular Geophila cordifolia and/or Geophila repens, in accordance with the invention is applied to at least a portion of the body, preferably a surface selected from the skin of the face, including the forehead, the contour of the lips and/or the contour of the eyes (pen-orbit), the oval of the face and the skin of the body including the neck, the hands, the arms, the thighs, the stomach, the hips and/or the bust, to stimulate the expression of LOXL and/or as a radical scavenger, anti-elastase agent and/or anti-MMP agent, in particular anti-MMP-2 and/or anti-MMP-12 and/or to increase quality and/or functionality of collagen fibres and/or to prevent and/or reduce unattractive and/or disagreeable and/or uncomfortable manifestations of aging, in particular of skin aging, and/or that are linked to an alteration in elastic and/or collagen fibres.

The present invention also relates to a food supplement in which the extract from the plant from the genus Geophila, in particular Geophila cordifolia and/or Geophila repens, in accordance with the invention is ingested orally to stimulate the expression of LOXL and/or as a radical scavenger, anti-elastase agent and/or anti-MMP agent, to increase quality and/or functionality of collagen fibres and/or to prevent and/or reduce unattractive and/or disagreeable and/or uncomfortable manifestations of aging, in particular of skin aging, and/or that are linked to an alteration in elastic and/or collagen fibres.

The composition of the invention is also intended for oral administration, in particular for “oral cosmetics”: it may in particular be in the form of capsules, gelules, dragees, granules, chewing gum, gels, potable syrups, tablets or any other form known to the skilled person. In particular, the extract of the invention may be incorporated into all forms of food supplements or enriched foods, for example nutrition bars, or compressed or other powders. The extract of the invention may be formulated with the usual excipients and components for such oral compositions or food supplements (in particular nutraceutics), namely fat and/or aqueous components, humectants, thickening agents, preservatives, texturizing agents, flavourings and/or coating agents, anti oxidants, preservatives and dyes which are usual in the food domain.

Other aims, characteristics and advantages of the invention will become apparent to the skilled person from the following explanatory description which refers to examples which are given solely by way of illustration and which in no way limit the scope of the invention.

The examples form an integral part of the present invention and any characteristic that appears novel over any prior art from the description taken as a whole, including the examples, forms an integral part of the invention in its function and in its generality.

Thus, each example has a general scope.

Moreover, in the examples, all of the percentages are given by weight unless otherwise indicated, the temperature is expressed in degrees Celsius unless otherwise indicated, and the pressure is atmospheric pressure unless otherwise indicated.

### EXAMPLES

### Example 1

### Preparation of an Extract of Geophila cordifolia According to the Invention

1a) An extract of Geophila cordifolia was obtained from aerial parts (stems and leaves) ground by maceration in water at 5% (w/w), at a temperature which was preferably between 0° C. and 20° C., preferably at 4° C.
The maceration period was advantageously between 30 min and 24 hours, with stirring, preferably 10 hours.
The solution was centrifuged, preferably for 10 min at 8000 rpm, and the supernatant was recovered. The supernatant was ultrafiltered on filters with different cutoff thresholds, in particular at 0.45 μm.
The extract obtained thereby was used directly in the liquid form.
1b) A Geophila cordifolia extract was obtained from aerial parts (stems and leaves) ground by maceration in a 75%/25% water/butylene glycol mixture, at a temperature which was preferably between 0° C. and 20° C., preferably 4° C. The maceration period was advantageously between 30 min and 24 hours, with stirring, preferably 10 hours.
The solution was centrifuged, preferably for 10 min at 8000 rpm, and the supernatant was recovered. The supernatant was ultrafiltered on filters with different cutoff thresholds, in particular at 0.45 μm.
The extract obtained thereby was then dried, in particular on a maltodextrin type support, then taken up into a 1% (w/w) solution in water.
1c) An extract of Geophila cordifolia was obtained from aerial parts (stems and leaves) ground by maceration in water at 1% (w/w), at a temperature which was preferably between 0° C. and 20° C., preferably at 20° C.
The maceration period was advantageously between 30 min and 24 hours, with stirring, preferably 2 hours.
The solution was centrifuged, preferably for 10 min at 8000 rpm, and the supernatant was recovered. The supernatant was ultrafiltered on filters with different cutoff thresholds, in particular at 0.45 μm.
The extract obtained thereby was used directly in the liquid form.
1d) An extract of Geophila cordifolia was obtained from aerial parts (stems and leaves) ground by maceration in water at 0.5% (w/w), at a temperature which was preferably between 0° C. and 20° C., preferably at 20° C.
The maceration period was advantageously between 30 min and 24 hours, with stirring, preferably 2 hours.
The solution was centrifuged, preferably for 10 min at 8000 rpm, and the supernatant was recovered. The supernatant was ultrafiltered on filters with different cutoff thresholds, in particular at 0.45 μm.
The extract obtained thereby was used directly in the liquid form.
In the following examples tested Geophila cordifolia extracts were obtained according to process 1a) or 1c). These extracts were then diluted in water or assay buffer or culture medium at the wished mentioned concentration.

### Example 2

### In Vitro Evaluation of the Radical Scavenger Effect of a Geophila Cordifolia Extract According to the Invention

### Principle:

The radical scavenger effect was evaluated:
using the standard test with DPPH (1,1-diphenyl-2-picrylhydrazyl) the principle of which is the measurement of the bleaching induced in the presence of anti oxidants;
by the lipid peroxidation method. The TBArs technique means that malondialdehyde (MDA), one of the terminal products formed during the decomposition of polyunsaturated fatty acids mediated by free radicals, can be assayed. It is assayed by condensation in an acidic medium with hot thiobarbituric acid (TBA), thereby forming a pink chromogen which absorbs strongly between 530 and 535 nm. The initial notion of MDA assay has gradually been substituted by the notion of thiobarbituric acid reactive substances (TBARS). The intensity of the colour is directly proportional to the quantity of MDA. The inhibition of UV-induced peroxidation during incorporation of an active agent allows its anti-oxidizing potential to be calculated.

### Experimental Protocol for TBARS Assay:

A 5% liposomal suspension was prepared in deionized water.
This suspension was incubated with the active agents to be tested or the positive control in 96-well plates. The 96-well plate was irradiated with UVA. A “blank” was also deposited on a non-irradiated control plate for the spectrophotometer reading (water), the non-irradiated positive control measurements and the non-irradiated test measurements (active agent tested at different concentrations).
The reaction was carried out directly in 96-well plates after the end of incubation.
1) a quantity of 100 μl of 20% TCA was added to each well (precipitation of lipids);
2) homogenization was carried out for 10 min using a plate agitator;
3) a quantity of 34 μl of 1% TBA 1% in 10% PCA was added;
4) the plates were covered and incubated for 25 minutes in an oven at 105° C.;
5) each sample was transferred to a multi-tube plate;
6) a centrifuging step was carried out for 5 minutes at 5700 rpm;
7) a quantity of 100 μl of supernatant was deposited in a new 96-well plate;
8) the optical density, OD, was read at 530 nm for the 96-well plate.
The percentage inhibition of UV-induced peroxidation is expressed using the following formula:

% inhibition=100−[(test measurement)/(100%−control 100%)×100]
blank: water
100%: liposomal suspension+solvent(s) for tested active agents—irradiated
100% control: liposomal suspension+solvent(s) for tested active agents—non-irradiated
tests: liposomal suspension+active agents at different test concentrations—irradiated
controls: liposomal suspension+active agents at different test concentrations—non-irradiated
   This means that the higher the measured percentage inhibition, the stronger the anti-oxidizing power of the active agent.
   The tested products were as follows:
the extract of the invention prepared in accordance with Example 1a) tested at final concentrations of 1% (w/w) and 3% (w/w);
a solution containing 2.5 mg/l or 5 mg/l of cysteine was used as a positive control in the DPPH test;
a 1% (w/w) solution of vitamin E was used as the positive control in the lipid peroxidation test;
negative control: solvent for extract.

### Results of DPPH Test:

The percentage inhibition was calculated for each test product with respect to the negative control (=100% of generated free radicals meaning 0% anti-free-radicals effect).

TABLE 1

| | anti -free -radicals effect | |
|---|---|---|
| Tested product | (% vs negative control) | Standard deviation |
| Cysteine 2.5 mg/l | 47.6 | 1.7 |
| Cysteine 5 mg/l | 83.1 | 2.7 |
| Extract 1a), 1% | 79.6 | 5.3 |

### Results of Lipid Peroxidation Test

The percentage inhibition was calculated for each test product with respect to the negative control (=100% of generated free radicals meaning 0% anti-free-radicals effect)

TABLE 2

| | anti -free -radicals effect | |
|---|---|---|
| Tested product | (% vs negative control) | Standard deviation |
| Vitamin E, 1% | 54.7 | 1.5 |
| Extract 1a), 1% | 10.0 | 0.5 |
| Extract 1a), 3% | 21.9 | 4.7 |

### Conclusions

The extract of the invention has anti-oxidizing properties. When tested at other concentrations (0.5 and 2%), it was also observed that this effect was dose dependant.

### Example 3

### In Vitro Evaluation of the Anti-Elastase Effect of an Extract of Geophila cordifolia According to the Invention

### Principle:

The anti-elastase activity was evaluated using HLE (Human Leucocyte Elastase). This test demonstrates the protective capacity of the test products against damage of the extracellular matrix by HLE. The principle of this test resides in measuring the degree of cleavage of a coloured peptide substrate by HLE.

### Experimental Protocol:

The tested products were as follows:
the extract of the invention was prepared in accordance with Example 1a) tested at final concentrations of 0.5%, 1%, 2% and 3% (w/w);
a 0.01% (w/w) solution of ursolic acid was used as the positive inhibition control for inhibition;
no solution was used as the negative control (the reaction buffer for the enzyme replaces the tested product).

### Results of HLE Test:

The percentage inhibition was calculated for each test product with respect to the negative control (=100% activity i.e. 0% inhibition).

TABLE 3

| Tested product | HLE inhibition (%) |
|---|---|
| Negative control | 0 |
| Ursolic acid, 0.01% | 85 |
| Extract 1a), 0.5% | 30 |
| Extract 1a), 1% | 45 |
| Extract 1a), 2% | 54 |
| Extract 1a), 3% | 58 |

### Conclusions

The extract of the invention has anti-HLE properties. This inhibition effect is also dosedependant.

### Example 4

### Analysis of Expression of LOXL in Presence of an Extract of Geophila cordifolia According to the Invention

### Principle:

The expression of genes for elastin and LOXL was measured using quantitative RT-PCR under standard culture conditions or after irradiation with increasing doses of UVB or UVA. Their level of expression has been related to the number of cells using the housekeeping gene actin. Expression of the LOXL gene was also followed by quantitative RT-PCR under non-irradiated conditions after application for 24 hours of a Geophila cordifolia extract obtained according to process 1a) at 1%.

### Experimental Protocol:

### 1) Culture Protocol:

Normal human fibroblasts (NH F) cultivated in a FGM medium (fibroblast growth medium, Promocell) were exposed to a UV stress.
A Fisher Bioblock type UVA or UVB source with an electric power of 0.3 mW/cm² was used. The UV dose was monitored using a radiometer (VLX 3W) and a CX-312 probe for UVB or a CX-365 probe for UVA.
Various UV irradiation conditions were evaluated:
For a wavelength of λ=312 nm (UVB)
UVB 5 mJ/cm², the exposure time was 30 seconds;
UVB 10 mJ/cm², the exposure time was 1 minute;

UVB 20 mJ/cm², the exposure time was 2 minutes.

For a wavelength of λ=365 nm (UVA)
UVA 0.1 J/cm², the exposure time was 4 minutes and 30 seconds;
UVA 1 J/cm², the exposure time was 48 minutes and 30 seconds;
UVA 7.5 J/cm², the exposure time was 6 hours and 53 minutes.
   After UV incubation cells are incubated for 24 hours at 37° C., 5% CO2 before molecular biological analysis.

### 2) Rna Extraction Protocol:

Total RNA from the monolayer culture was extracted with the SV96 Total RNA Isolation System® following the manufacturer's instructions. The total RNA was eluted in 100 μl of nuclease-free water and quantified using a spectrophotometer at 260 nm (Molecular Devices Spectramax 190). The total RNA was distributed directly into the wells of RT-PCR plates in an amount of 50 ng per well and maintained at −80° C.

### 3) RT-PCR Protocol

A quantity of 50 ng of total RNA was amplified by Real Time RT-PCR (Quantitek Sybr Green® kit). The primers used in the RT-PCR experiments (semi-quantitative or real time) were as follows:

TABLE 4

| | Length of | | |
|---|---|---|---|
| | amplified | | |
| Gene | DNA (bp) | Primer | sequence |
| Actin | 540 | Sense | 5′-gtg ggg cgc ccc agg cac ca-3′ |
| | | Anti-sense | 5′-ctc ctt att gtc acg cac gat ttc-3′ |
| Elastin | 357 | Sense | 5′-gtg tat acc cag gtg gcg tg-3′ |
| | | Anti-sense | 5′-cga act ttg ctg ctg ctt tag-3′ |
| LOXL | 240 | Sense | 5′-gac ttc ggc aac ctc aag c-3′ |
| | | Anti-sense | 5′-tgt tgc aga aac gta gcg ac-3′ |

The amplification reaction was carried out using a thermocycler (Opticon 1® (MJ Research)). The PCR products were analysed on agarose gel to verify the pertinence of the fluorescence data, i.e. the presence of a single fluorescence band at the expected size) and the PCR products were sequenced by MWG Biotech (Germany). Quantification was carried out using the ΔCt method for the other experiments. Expression of the gene was normalized with respect to the expression of the gene for actin.

### 4) Statistical Analyses:

For each sample, the experiment was repeated at least 3 times and the differences between the means were determined using a One way Anova test. The difference was considered to be significant when p<0.05.

### 5) Treatment Protocol

The NHFs were treated or not treated with the extract of the invention in an amount of 1% in the culture medium for 24 hours. After 24 hours, the cell layers were removed for the molecular biological analyses described above.

### Results:

### Modulation of the Expression of Genes Referred to Actin Under the Action of UV in the Presence or Absence of the Agent of the Invention.

TABLE 5
- Gene modulation under UVA

| | Gene/actin | Mean | Std deviation |
|---|---|---|---|
| Non-irradiated ELN | 0.754 | 1 | 0.26 |
| | 1.305 | | |
| | 1.217 | | |
| | 0.724 | | |
| 0.1 J/cm² ELN | 1.923 | 1.35 | 0.45 |
| | 1.674 | | |
| | 1.074 | | |
| | 0.709 | | |
| 1 J/cm² ELN | 0.58 | 1.59 | 0.84 |
| | 2.076 | | |
| | 2.777 | | |
| | 0.929 | | |
| 7.5 J/cm² ELN | 9.67 | 7.69 | 2.47 |
| | 5.218 | | |
| | 10.656 | | |
| | 5.218 | | |
| Non-irradiated LOXL | 1.215 | 1 | 0.27 |
| | 0.612 | | |
| | 1.32 | | |
| | 0.853 | | |
| 0.1 J/cm² LOXL | 1.396 | 1.18 | 0.14 |
| | 1.232 | | |
| | 1.19 | | |
| | 0.896 | | |
| 1 J/cm² LOXL | 1.614 | 1.56 | 0.2 |
| | 1.517 | | |
| | 1.906 | | |
| | 1.19 | | |
| 7.5 J/cm² LOXL | 0.865 | 0.9 | 0.07 |
| | 0.836 | | |
| | 1.029 | | |
| | 0.865 | | |

TABLE 6
- Gene modulation under UVB

| | Gene/actin | Mean | Std deviation |
|---|---|---|---|
| Untreated, non-irradiated | 1.050908 | 1 | 0.13 |
| ELN | 1.058218 | | |
| | 1.158001 | | |
| | 0.732874 | | |
| IL-1b ELN | 9.393332 | 14.74 | 6.87 |
| | 11.016828 | | |
| | 28.475258 | | |
| | 10.067524 | | |
| UVB 5 mJ/cm² ELN | 1.190557 | 1.05 | 0.2 |
| | 0.612014 | | |
| | 0.980532 | | |
| | 1.406039 | | |
| | 1.043649 | | |
| UVB 10 mJ/cm² ELN | 1.095536 | 1.35 | 0.44 |
| | 2.221658 | | |
| | 0.99422 | | |
| | 1.07299 | | |
| UVB 20 mJ/cm² ELN | 1.76741 | 1.57 | 0.33 |
| | 1.142059 | | |
| | 2.175937 | | |
| | 1.592879 | | |
| | 1.182333 | | |
| untreated non-irradiated | 1.146902 | 1 | 0.14 |
| LOXL | 1.019415 | | |
| | 0.725849 | | |
| | 1.107834 | | |
| IL-1b LOXL | 1.107834 | 1.12 | 0.07 |
| | 0.977888 | | |
| | 1.138979 | | |
| | 1.246378 | | |
| UVB 5 mJ/cm² LOXL | 1.738378 | 1.38 | 0.16 |
| | 1.345126 | | |
| | 1.272567 | | |
| | 1.441671 | | |
| | 1.123299 | | |
| UVB 10 mJ/cm² LOXL | 0.957764 | 1.07 | 0.1 |
| | 1.107834 | | |
| | 1.012374 | | |
| | 1.281418 | | |
| | 0.991539 | | |
| UVB 20 mJ/cm² LOXL | 1.281418 | 1.42 | 0.11 |
| | 1.61076 | | |
| | 1.461796 | | |
| | 1.471963 | | |
| | 1.272567 | | |

Treatment of fibroblasts with Geophila Cordifolia extract;
The results are expressed as the induction of the LOXL gene with respect to the control not treated with extract 1a) at 1%.

TABLE 7

| LOXL/actin ratio | Mean | Standard deviation |
|---|---|---|
| 4.8568 | 2.94 | 1.28 |
| 2.1287 | | |
| 2.3784 | | |
| 2.4116 | | |

### Conclusions

These results demonstrate that UVA induces a greater increase in damage than UVB. In particular, UVA induces a very substantial increase in the expression of the tropoelastin gene in a dose-dependent manner. However, since this is not correlated to an increase in the expression of the LOXL gene, the result is an absence of appropriate cross-linking of the tropoelastin synthesized thereby into functional fibres. Elastogenesis is thus non-functional, especially as degradation of the tropoelastin thus synthesized increases in parallel under the action of the MMPs induced by the UV. The elastic fibres are thus disorganized, a phenomenon characteristic of solar elastosis.
This demonstrates the advantage of an active agent which stimulates the expression of LOXL in order to prevent and/or combat the deleterious effects of UV, in particular UVA.
The extract in accordance with the invention increased the expression of the gene for LOXL since the mRNA synthesis of LOXL is almost triple. This provides evidence of the effectiveness of the extract of the invention on increasing the expression of LOXL and its capacity for being used as a particularly advantageous agent for preventing and/or combating alterations in the elastic fibres.

### Example 5

### In Vitro Evaluation of Anti-MMP Effect of an Extract of Geophila cordifolia According to the invention

### Principle:

Anti-MMP activity was evaluated on matrix metalloproteases MMP-2 and MMP-12. This test demonstrates the ability of the tested products to protect extracellular matrix against damages by MMPs and is based on kinetic measurement of the transformation of a peptidic substrate into coloured product.

### Experimental Protocol:

The substrat of MMPs is thiopeptide [Ac-PLG-2mercapto-4-methyl-pentanoyl)-LG-OC2H5] (EnzoLifeSciences, P125-9090). The test is carried out in 96-well plates. Different medium are prepared:
A <<blank>> containing assay buffer and the substrate—Value obtained with blank was deduced from all other conditions.
Positive control, NNGH (EnzoLifeSciences, PI 115-9090), which is metalloproteinase inhibitor, diluted at 1/200 in assay buffer.
Untreated control, containing assay buffer, MMP and the substrate: this corresponds to 100% MMP activity.

Other conditions contain assay buffer, MMP, the substrate and the solution to be tested. After 45 min preincubation at 37° C. (to get interaction between MMP and tested product: tested solution or positive control), substrate is added and plate is immediately placed in Plate reader for kinetic measurement of absorbance at 405 nm (maximum absorbance of coloured product resulting from degradation of substrate by MMP). Full plate is read each minute during 20 minutes. After 20 min, a curve reflecting kinetic evolution of MMPs activity is obtained.

In the linear phase, the slope of the curve provides with MMP activity.
The percentage inhibition was calculated with respect to the untreated control according to the formula:

Inhibition percentage=100−((slope of tested product/Mean of the untreated control slopes)*100)

This test was performed on MMP-2 and MMP-12.
Tested solution containing Geophila cordifiolia extract was obtained according to example 1c) and tested at different concentration.

### Results:

### Statistical Analyses:

+=p<0.05, +++=p<0.001 (T-test); x=p<0.05, xx=p<0.01, xxx=p<0.001 (Anova on Ranks)

| MMP-2 | Mean (%) | Standard deviation |
|---|---|---|
| Untreated control | 0.00 | 10.25 |
| Positive control (NNGH) | 73.43⁺⁺⁺ | 4.31 |
| Extract 1c) 0.5% | 12.32 | 4.81 |
| Extract 1c) 1% | 30.52^{xx} | 10.14 |
| Extract 1c) 2% | 38.29^{xxx} | 4.01 |
| Extract 1c) 3% | 35.74^{xx} | 2.91 |

| MMP-12 | Mean (%) | Standard deviation |
|---|---|---|
| Untreated control | 0.00 | 17.16 |
| Positive control (NNGH) | 58.63⁺⁺⁺ | 21.04 |
| Extract 1c) 0.50% | 10.52 | 13.97 |
| Extract 1c) 1% | 63.76^{x} | 8.63 |
| Extract 1c) 2% | 59.59^{xx} | 6.25 |
| Extract 1c) 3% | 44.24^{xx} | 9.08 |

### Conclusion:

Inhibition activity which is below 25% is considered as non significant and inhibition activity which is more than 50% is considered to be high.
It is observed that positive control (NNGH) strongly and significantly inhibits activity of all tested MMPs. Moreover, inhibitory activity of MMP-2 and MMP-12 is observed with extract 1a) at 1% and higher concentrations. Maximal significant inhibitory effect is observed at 2%.

### Example 6

### In Vitro Evaluation of the Effect of an Extract of Geophila cordifolia According to the Invention on the Organization of Type I Collagen Fibres

### Experimental Protocol:

Immunofluorescence makes it possible to observe different markers expressed by cells via the use of antibody recognizing these markers.
In first step, cells were fixed in cold methanol for 10 minutes. After washing with PBS, antigenic aspecific sites were then blocked for 1 hour with PBS-BSA 1% solution (Bovine serum albumin). Primary antibody diluted at 1% in PBS/BSA 1% was incubated with cells for 1 hour. On the negative control is added IgG control isotope (instead of primary antibody). The excess of anti-body which has not been attached is removed by 2 washings with PBS.
Cells were incubated out of light with a solution of secondary antibodies able to recognize primary antibody. This secondary antibody being linked with fluorochrome (Alexa Fluor 488), it makes it possible to see protein with florescent microscopy. Cells were then gently washed 3 times with PBS, and slides covered with mounting medium containing DAPI (Vector®, Vectashield), a nuclear colouring agent. Slides were stored out of light to maintain fluorescence of fluorochrome and analysed with confocal microscopy.
Antibodies used in this test were:
primary antibody: collagen 1: rabbit anti-human type I collagen, polyclonal, diluted 1/200 (Novotech 20111)
primary antibody: Isotype IgG1: rabbit IgG1 isotype, ready to use (Zymed Laboratories 08-6199)
secondary antibody: goat anti-rabbit IgG coupled with Alexa fluor 488, polyclonal, diluted 1/1000 (in Vitrogen A11008)
   Cells used for immunostaining were obtained from fibroblasts from donor aged 61 years old. Geophila cordifolia obtained according to example 1c) was tested at 1% (diluted in water).

### Results:

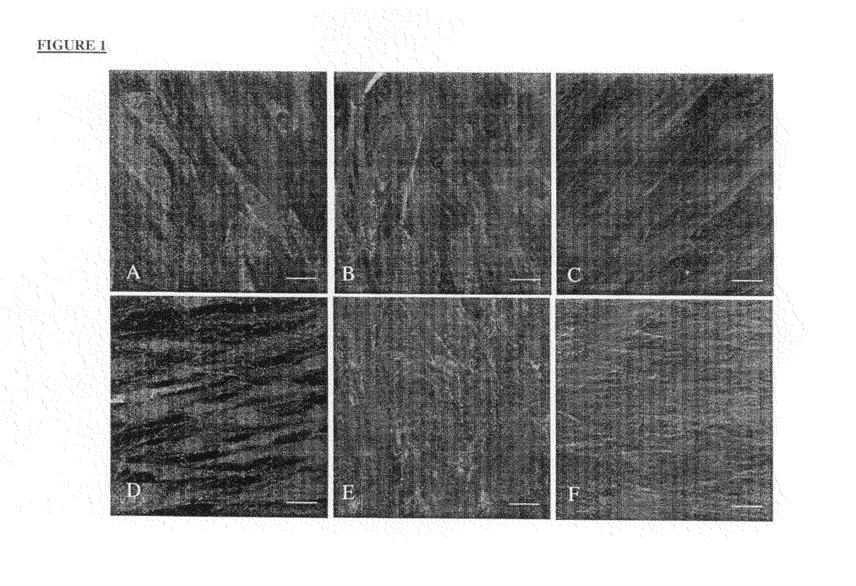

### FIG. 1:

Fluorescent Immunostaining of type I collagen on monolayer cultured fibroblasts treated with vegetal extract obtained from example 1c) (magnification 400×, scale bar=25 μm).

### A Untreated Fibroblasts—day 3.

B Fibroblasts treated with Geophila cordifolia 1c) at 1%-day 3
C Fibroblasts treated with vitamin C at 50 μg/ml-day 3

D Untreated Fibroblasts—day 5

E Fibroblasts treated with Geophila cordifolia 1c) at 1%-day 5
F Fibroblasts treated with vitamin C at 50 μg/ml-day 5

After 3 days, mainly intracellular and cytoplasmic labelling was observed on untreated cells (picture A). When treated with vitamin C, a fibrillar labelling, being more external to the cell was observed (picture C) and high anisotropy could be seen ie fibres were all oriented in the same direction, parallel to each others. Concerning cells treated by Geophila cordifolia, observed labelling was also fibrillar and extracellular (picture B) but collagen type I fibres were thicker and orientated in all directions ie anisotropy was decreased with extract according to the invention.

After 3 days treatment, booster effect on collagen type I is thus already observed with Geophila cordifolia extract compared with not treated control and is stronger than effect observed with vitamin C. Vitamin C is a well know referent for its positive effects on polymerization of collagen fibres and collagen synthesis.
After 5 days, type I collagen in untreated fibroblasts was still observed in cell cytoplasm (picture D) whereas it was observed much more extracellular collagen fibres when cells were treated 5 days with vitamin C or Geophila cordifolia.
Fibroblasts treated with Geophila cordifolia had more fibrillar collagen than untreated fibroblasts, thus forming important and stronger matrix network of intercrossing fibres and fibrils which means that anisotropy was decreased. Labelling is fully extracellular (picture E). With cells treated with vitamin C, it is observed that labelling is not so intense and fibres are more oriented in one way and thinner which means that anisotropy is increased with Vitamin C. (picture F)

### Conclusion:

Extract of Geophila cordifolia according to the invention stimulates organization of collagen type

I fibres in matrix. Contrary to vitamin C, extract of Geophila cordifolia does not induce deposit of parallels fibres, which is more in line with physiology and functionality of collagen fibres. This proves that extract according to the invention increases quality and functionality of collagen fibres, in particular type 1 collagen notably as it decreases anisotropy of collagen fibres.

### Example 7

### Preparation of an Extract of Geophila repens in Accordance with the Invention

7a) An extract of Geophila repens was obtained from aerial parts (stems and leaves) ground by maceration in water at 5% (w/w), at a temperature which was preferably between 0° C. and 20° C., preferably at 4° C.
The maceration period was advantageously between 30 min and 24 hours, with stirring, preferably 10 hours.
The solution was centrifuged, preferably for 10 min at 8000 rpm, and the supernatant was recovered. The supernatant was ultrafiltered on filters with different cutoff thresholds, in particular at 0.45 μm.
The extract obtained thereby was used directly in the liquid form.
7b) A Geophila repens extract was obtained from aerial parts (stems and leaves) ground by maceration in a 75%/25% water/butylene glycol mixture, at a temperature which was preferably between 0° C. and 20° C., preferably 4° C. The maceration period was advantageously between 30 min and 24 hours, with stirring, preferably 10 hours.
The solution was centrifuged, preferably for 10 min at 8000 rpm, and the supernatant was recovered. The supernatant was ultrafiltered on filters with different cutoff thresholds, in particular at 0.45 μm.
The extract obtained thereby was then dried, in particular on a maltodextrin type support, then taken up into a 1% (w/w) solution in water.
7c) An extract of Geophila repens was obtained from aerial parts (stems and leaves) ground by maceration in water at 1% (w/w), at a temperature which was preferably between 0° C. and 20° C., preferably at 4° C.
The maceration period was advantageously between 30 min and 24 hours, with stirring, preferably 10 hours.
The solution was centrifuged, preferably for 10 min at 8000 rpm, and the supernatant was recovered. The supernatant was ultrafiltered on filters with different cutoff thresholds, in particular at 0.45 μm.
The extract obtained thereby was used directly in the liquid form.
7d) An extract of Geophila repens was obtained from aerial parts (stems and leaves) ground by maceration in water at 0.5% (w/w), at a temperature which was preferably between 0° C. and 20° C., preferably at 20° C.
The maceration period was advantageously between 30 min and 24 hours, with stirring, preferably 2 hours.
The solution was centrifuged, preferably for 10 min at 8000 rpm, and the supernatant was recovered. The supernatant was ultrafiltered on filters with different cutoff thresholds, in particular at 0.45 μm.
The extract obtained thereby was used directly in the liquid form.
In following examples, extracts obtained according to process 7a) or 7c) were used. These extracts were dissolved in water or assay buffer or culture medium at the wished final mentioned tested concentration.

### Example 8

### In Vitro Evaluation of Efficacy of Geophila Repens

### Experimental Protocol:

Tests used were performed in the same conditions than above described with Geophila cordifolia.
Extract of Geophila repens was obtained according to example 7a) and tested at different concentrations.

### Results:

radical scavenging activity (DPPH test):

| | | Positive | Positive | | | |
|---|---|---|---|---|---|---|
| | Negative | control | control | | | |
| | control | (cysteine) | (cysteine) | Geophila repens 7a) | | |
| con- | | 2.5 mg/l | 5 mg/l | ″0.5% | 1% | 2.00% |
| centration | | | | | | |
| Mean | 0.00 | 68.52 | 79.49 | 52.78 | 80.85 | 89.60 |
| Standard | 5.11 | 4.46 | 4.76 | 3.23 | 5.36 | 1.84 |
| deviation | | | | | | |

anti-elastase activity (HLE test):

| | Negative | Negative | | | | |
|---|---|---|---|---|---|---|
| | control | control | Positive | Geophila repens 7a) | | |
| | DMSO | water | control* | 2.00% | 1.00% | 0.50% |
| mean | 0.00 | 0.00 | 90.47 | 56.66 | 45.32 | 34.60 |
| Standard | 11.26 | 5.11 | 2.32 | 5.03 | 4.42 | 5.58 |
| deviation | | | | | | |

*Positive control: ursolic acid 0.01%

gene expression of LOXL: (RT-PCR)

| | mean | Standard deviation |
|---|---|---|
| Negative control | 1 | 0.14 |
| Geophila repens 7a) at 1% | 1.97 | 0.21 |

anti-MMP2 effect:

| | Negative | Positive control | Geophila repens 7a) | | |
|---|---|---|---|---|---|
| | control | (NNGH) | 3% | 2% | 1% |
| mean | 0.00 | 62.51 | 113.00 | 89.62 | 84.78 |
| Standard deviation | 32.10 | 40.87 | 3.84 | 18.01 | 19.27 |

anti-MMP 12 effect:

| | Negative | Positive control | Geophila repens 7a) | | |
|---|---|---|---|---|---|
| | control | (NNGH) | 3% | 2% | 1% |
| mean | 0.00066667 | 72.5825 | 41.786 | 100.071 | 75.473 |

### Conclusion:

Geophila repens also has following properties:
antioxidant
anti-elastase activity
stimulation of expression of LOXL this combating alterations of elastic fibres, in particular UV induced.

anti-MMP-2 and anti-MMP12 effect

### Example 8

### Examples of Producing Compositions in Accordance with the Invention

Methods known to the skilled person were used to mix together the various portions A, B, C, D, E, or F in order to prepare a composition in accordance with the present invention.
The “products of the invention” preferably represent an extract of Geophila cordifolia obtained according with example 1d) or an extract of Geophila repens obtained according with example 7d).
The products of the invention may also be in the form of liposomes containing 5% of soya lecithin and incorporating a quaternized soya solution (600 g final) obtained using the following procedure:
30 g of soya lecithin, 12 g of quaternized soya solution, 1.5 g of 1.5 g of Geophila cordifolia extract prepared in accordance with Example 1d) or 1.5 g of 1.5 g of Geophila repens extract prepared in accordance with Example 7d) were introduced into a pill machine and diluted in 447 g of laboratory pure water.
After stirring magnetically for 10 minutes at ambient temperature, the mixture was homogenized violently for 10 minutes, to obtain thereby a liposomal solution in which the liposomes had a mean dimension which could vary between 100 and 800 nanometres depending on the exact homogenization conditions.
The suspension was then stirred gently for 1 hour. 90 g of butylene glycol, 6 g of phenoxyethanol and 6 g of hydroxyethylcellulose (gelling agent) were then added.

### Formulation 8a:

| A | Water | qsp 100 |
|---|---|---|
| | Butylene Glycol | 2 |
| | Glycerin | 3 |
| | Sodium Dihydroxycetyl Phosphate, | 2 |
| | Isopropyl Hydroxycetyl Ether | |
| B | Glycol Stearate SE | 14 |
| | Triisononaoin | 5 |
| | Octyl Cocoate | 6 |
| C | Butylene Glycol, Methylparaben, | 2 |
| | Ethylparaben, Propylparaben, | |
| | pH adjusted to 5.5 | |
| D | Products of the invention | 0.01-10% |

### Formulation 8b:

| A | Water | qsp 100 |
|---|---|---|
| | Butylene Glycol | 2 |
| | Glycerin | 3 |
| | Polyacrylamide, Isoparaffin, Laureth-7 | 2.8 |
| B | Butylene Glycol, Methylparaben, | 2 |
| | Ethylparaben, Propylparaben; | |
| | Phenoxyethanol, Methylparaben, | 2 |
| | Propylparaben, Butylparaben, Ethylparaben | |
| | Butylene Glycol | 0.5 |
| D | Products of the invention | 0.01-10% |

### Formulation 8c:

| | | |
|---|---|---|
| A | Carbomer | 0.50 |
| | Propylene Glycol | 3 |
| | Glycerol | 5 |
| | Water | qsp 100 |
| B | Octyl Cocoate | 5 |
| | Bisabolol | 0.30 |
| | Dimethicone | 0.30 |
| C | Sodium Hydroxide | 1.60 |
| D | Phenoxyethanol, Methylparaben, | 0.50 |
| | Propylparaben, Butylparaben, Ethylparaben | |
| E | Fragrance | 0.30 |
| F | Products of the invention | 0.01-10% |

1. A cosmetic, dermatological, food supplement or pharmaceutical composition, comprising a plant extract from the genus Geophila, preferably from Geophila cordifolia or Geophila repens.

2. The composition of claim 1, further comprising a vehicle suitable for cosmetic, dermatological, food supplement or pharmaceutical use.

3. The composition of claim 1, comprising another ingredient which is active cosmetically, dermatologically, as a food supplement or pharmaceutically.

4. The pharmaceutical composition of claim 1, for preventing and/or treating at least one pathology linked to skin aging and/or to a loss of functionality of elastic and/or collagen fibres, in particular in the case of the skin, fibrosis, scleroderma, atherosclerosis, emphysema, pseudo-xanthoma elasticum (PXE), hypertrophic, keloid and/or dystrophic scars and/or associated with a genetic deficiency affecting the elastic fibres, such as Marfan syndrome, progeria, cutis laxa, genetic solar elastosis, dystrophic, keloid and/or hypertrophic scars and/or pathologies linked to vascular tissue deficiency, and/or intended to prevent and/or treat pathologies due to an anti-oxidizing stress, such as cardiovascular diseases, atherosclerosis, diabetes, neuropathies or cancer, and/or intended to prevent and/or treat pathologies due to elastases and/or metalloproteinases, in particular elastolysis affecting pulmonary tissue, arteries, gums and skin, especially due to diseases linked to a deficiency in elastase and/or metalloproteinase inhibitors, in particular serpins, cystatins and TIMPs (tissue inhibitor of metalloproteinases), varicose veins and inflammations, gingivitis and periodontitis, psoriasis, dermatitis and eczema, and sensitive skin.

5. The composition of claim 1, wherein the plant extract is at a concentration between 1×10⁻⁴ and 10% by weight, advantageously between 1×10⁻⁴ and 5% of the total composition weight.

6. The composition of claim 1, wherein the plant extract is at a concentration between 1×10⁻³ and 3% of the total composition weight.

7. The composition of claim 1, wherein the plant extract is obtained from the aerial parts of the plant, in particular stem, leaves, and/or a mixture thereof.

8. A method for stimulating expression of LOXL and/or for increasing quality and/or functionality of collagen fibres, in particular type I collagen fibres, comprising utilizing a plant extract from the genus Geophila, preferably from Geophila cordifolia and/or Geophila repens as an agent, an anti-oxidizing agent, an anti-elastase agent, and/or an anti-MMP agent.

9. The method of claim 8, wherein the plant extract is used as an anti-MMP agent, and wherein the MMP is MMP-2 and/or MMP-12.

10. A method for preparing a cosmetic, dermatological, food supplement or pharmaceutical composition, comprising utilizing a plant extract from the genus Geophila, preferably from Geophila cordifolia and/or Geophila repens.

11. The method of claim 10, wherein the plant extract from the genus Geophila, preferably Geophila cordifolia and/or Geophila repens, is used for preparing a pharmaceutical composition, especially a dermatological composition, for preventing and/or treating at least one pathology linked to aging in a human being, in particular linked to skin aging and/or an alteration in elastic fibres and/or alteration in collagen fibres.

12. The method of claim 8, wherein the plant extract from the genus Geophila, preferably Geophila cordifolia and/or Geophila repens, is used to produce a food supplement composition to prevent and/or combat aging in a human being, in particular skin aging and/or an alteration in elastic fibres and/or alteration in collagen fibres.

13. The method of claim 8, wherein the plant extract from the genus Geophila, preferably Geophila cordifolia and/or Geophila repens, is used in a cosmetic composition to prevent and/or combat aging in a human being, in particular skin aging and/or an alteration in elastic and/or collagen fibres, or in an anti-wrinkle or anti-aging composition.

14. The method of claim 8, wherein the plant extract from the genus Geophila, preferably Geophila cordifolia and/or Geophila repens, is used in a cosmetic composition for sensitive and/or irritated skin, or in a hair composition, in particular for anti-hair loss or for hair regrowth, or in a product for making up the skin of the face, body or lips such as a foundation, a lipstick, an oral hygiene product such as a toothpaste or a mouthwash lotion; or in a sunscreen and/or after-sun composition.

15. The method of claim 8, wherein the composition is applied topically or administered orally.

16. A cosmetic skin care method comprising topically applying or orally administering the composition of claim 1 or a plant extract from the genus Geophila, preferably Geophila cordifolia and/or Geophila repens, to prevent and/or reduce and/or retard an alteration in elastic and/or collagen fibres, loss of resistance, elasticity and/or flexibility and/or firmness in tissues, and in particular the skin, and/or to prevent and/or reduce unattractive and/or disagreeable and/or uncomfortable manifestations of aging, in particular in the skin, and/or to stimulate the expression of LOXL, and/or as a radical scavenger, anti-elastase agent and/or anti-MMP agent, and/or increase quality and/or functionality of collagen fibres and/or to prevent and/or reduce unattractive and/or disagreeable and/or uncomfortable manifestations of skin aging.
